(19) United States
(12) Patent Application Publication
(10) Pub. No.: US 2014/0228654 A1
(43) Pub. Date: Aug. 14, 2014

(54) DIAGNOSTIC MEASUREMENT DEVICE

(71) Applicant: Ingo FLORE, (US)

(72) Inventors: Yoon Ok Kim, Schwerte (DE); Ok Kyung Cho, Schwerte (DE)

(73) Assignee: Ingo FLORE, Dortmund (DE)

(21) Appl. No.: 14/346,280

(22) PCT Filed: Sep. 21, 2012
(86) PCT No.: PCT/EP2012/003957

§ 371 Date: Mar. 20, 2014

(30) Foreign Application Priority Data
Sep. 21, 2011 (DE) 10 2011 113 841.6

(51) Int. Cl. A61B 5/0205; A61B 5/145; A61B 5/00; A61B 5/0408; A61B 5/03; A61B 5/01

(52) U.S. Cl. CPC A61B 5/02055; A61B 5/03; A61B 5/01; A61B 5/4866; A61B 5/0059; A61B 5/04085; A61B 5/14532; A61B 5/7278; A61B 5/021
USPC 600/301; 600/561

(57) ABSTRACT

Measurement device and method for noninvasive determination of at least one physiological parameter, the device comprising a diagnostic sensor unit (1) for generating measurement signals, and an evaluation unit for processing the measurement signals. The diagnostic sensor unit (1) comprises at least one pressure sensor (3) which detects the pressure exerted locally onto the pressure sensor (3) by a body tissue which is to be examined. The evaluation unit is configured to derive at least one physiological parameter from the measurement signal of the pressure sensor (3).

The invention relates to a measurement device for non-invasive determination of at least one physiological parameter, having a diagnostic sensor unit for generating measurement signals, and having an evaluation unit for processing the measurement signals. Furthermore, the invention relates to a method for non-invasive determination of at least one physiological parameter.

A measurement device of the type mentioned above is known, for example, from WO 2008/061788 A1. The previously known measurement device has a sensor unit that is integrated into the keyboard of a computer or into a mobile device of entertainment or communications technology. In this connection, the diagnostic sensor unit comprises different measurement modalities, namely an optical measurement unit, an EKG unit, a temperature sensor and/or a bioelectrical impedance unit. The combination of the different measurement modalities allows combined evaluation of the corresponding measurement signals by means of an evaluation unit programmed in suitable manner. In this connection, the combination guarantees great efficiency and reliability in the recognition of pathological disturbances. In particular, the previously known measurement device allows non-invasive, indirect measurement of metabolic parameters. Ultimately, non-invasive measurement of the glucose concentration and of the blood glucose level is possible with this.

It is the task of the invention to make available a measurement device having expanded functionality as compared with the state of the art. It is furthermore the task of the invention to make available a measurement device having a diagnostic sensor unit having the simplest possible structure, so that the measurement device as a whole can be produced in cost-advantageous manner.

To accomplish at least one of the said tasks, the invention proposes, proceeding from a measurement device of the type mentioned initially, that the diagnostic sensor unit comprises at least one pressure sensor that detects the pressure exerted locally on the pressure sensor by a body tissue to be examined, whereby the evaluation unit is set up for derivation of at least one physiological measurement signal from the measurement signal of the pressure sensor.

The pressure sensor provided in the measurement device according to the invention detects a uniaxial pressure. To put it differently, the pressure sensor detects a force exerted on the pressure sensor by the body tissue, in a specific direction, generally in the direction perpendicular to the body surface at which the pressure sensor makes contact. Therefore, a force sensor is also understood to be a pressure sensor in the sense of the invention. According to the invention, the pressure exerted locally on the pressure sensor by the body tissue to be examined is detected. In this connection, it is assumed that the pressure sensor utilizes a (comparatively small) contact surface that lies against the body surface and by way of which the pressure exerted by the body tissue is detected. The size of the contact surface can lie in the range from 1 mm² to 10 cm², for example. The pressure locally exerted on the pressure sensor is the pressure detected by way of this contact surface of the pressure sensor, in the sense of the invention.

The above-cited document WO 2008/061788 A1 already mentions pressure sensors that serve, however, in the previously known device, to detect the press-down pressure of a finger of a user of the device, because the press-down pressure influences the measurement signals of the other measurement modalities. The finger press-down pressure that is determined is taken into consideration in the evaluation of the measurement signals, in order to compensate the influence of the press-down pressure. In contrast to the invention, the measurement signals of the pressure sensor in the previously known device therefore do not serve for deriving at least one physiological parameter from the measurement signal of the pressure sensor.

It is therefore the core of the invention to use the pressure sensor as a diagnostic sensor. For example, the evaluation unit of the measurement device according to the invention can be set up for deriving at least one of the following physiological parameters from the time progression of the measurement signal of the pressure sensor: pressure volume pulse, pulse amplitude, pulse width, pulse duration, perfusion, blood pressure, pulse pressure, heart rate, pulse wave velocity, body temperature, metabolically generated heat. The progression of the measurement signal of the pressure sensor directly yields the pressure volume pulse. From this, various other physiological parameters can then be determined. For example, the pulse amplitude correlates with the body temperature, so that, preferably after corresponding calibration, the body temperature, namely the body surface temperature and/or the average body temperature, the arterial blood temperature, the body core temperature or the metabolically produced amount of heat, can be derived from the measurement signal of the pressure sensor. Furthermore, conclusions can be drawn concerning the amount of heat that flows from the body interior to the end of the arterial capillaries, and concerning the temperature in the region of the arterial-venous capillaries or the body tissue surrounding them.

To obtain the measurement signal by means of the measurement device according to the invention, the at least one pressure sensor must be brought into contact with the body surface of the user, directly or indirectly, for example in that the user lays a finger onto the device equipped with the pressure sensor. Likewise, it is possible that the pressure sensor is pressed against the surface of the body tissue, using suitable means. A holding clip of a known type or an elastic cuff can be used for this purpose. Likewise, it is possible that the measurement device with the pressure sensor is guided along the body of the user, for example by medically trained personnel, in order to measure the pressure at different locations of the body. After the pressure sensor is laid against the body of the user, the measurement signal is detected over a predetermined period of time (several seconds to several minutes, or also continuously). The time progression of the measurement signal is then evaluated as described above.

In a preferred embodiment, the sensor unit of the measurement device according to the invention has an optical measurement unit that comprises at least one radiation source for irradiation of the body tissue and at least one radiation sensor for detection of the radiation scattered and/or transmitted by the body tissue, whereby the evaluation unit is set up for derivation of the at least one physiological parameter at least from the measurement signal of the pressure sensor and of the optical measurement unit. The optical measurement unit can be equipped as described in the above-cited WO 2008/061788 A1. For example, the arterial oxygen saturation can be determined by means of the optical measurement unit. In this connection, the pressure volume pulse determined by means of the pressure sensor can supplement or partly replace the optical measurement, because the pressure volume pulse can be determined independent of the light absorption of individual body tissue or blood components. Thus, the measurement signal of the pressure sensor can serve as a reference value and thereby increase the measurement accuracy. Furthermore, detection of the measurement signal of the pressure sensor allows simplification of the optical measurement. For example, the measurement can be reduced to a small number of different wavelengths.

According to another preferred embodiment, the sensor unit of the measurement device comprises a temperature sensor, whereby the evaluation unit is set up for derivation of the at least one physiological parameter at least from the measurement signals of the pressure sensor and of the temperature sensor. From the cited WO 2008/061788 A1, it is known that the glucose concentration in the blood can be determined from a combined temperature and optical measurement. Accordingly, the temperature or heat measurement can supplement the further measurement modalities in practical manner. The measurement signals of the temperature sensor allow conclusions concerning the local heat exchange and thereby concerning the local metabolic activity. Furthermore, the temperature sensor is suitable for determining the local perfusion.

In another preferred embodiment, the sensor unit of the measurement device according to the invention has an EKG unit for detection of the EKG signal by way of two or more EKG electrodes, whereby the evaluation unit is set up for derivation of the at least one physiological parameter at least from the measurement signals of the pressure sensor and of the EKG unit. The functional scope of the measurement device according to the invention is expanded by means of the EKG unit. The evaluation unit of the measurement device can be set up, for example, for evaluation of the progression of the pressure volume pulse and of the EKG signal over time. From this, in turn, it is possible to determine the pulse wave velocity, which allows conclusions concerning the blood pressure, among other things. To put it differently, the combination of pressure sensor and EKG unit makes automated functional evaluation of the state of the vascular system of the user of the measurement device possible.

In another preferred embodiment, the sensor unit of the measurement device according to the invention has a bioelectrical impedance measurement unit, whereby the evaluation unit is set up for derivation of the at least one physiological parameter at least from the measurement signal of the pressure sensor and of the bioelectrical impedance measurement unit. The bioelectrical impedance measurement unit can be configured, for example, as in the above-cited WO 2008/061788 A1. In particular, the bioelectrical impedance measurement unit can be configured for local bioimpedance measurement. The combination of pressure sensor and bioelectrical impedance measurement unit allows the determination of the amount of water contained in the body tissue, of the proportion of the fat-free mass of the body tissue, and of the body fat proportion, without the total body mass necessarily having to be determined or entered. The bioelectrical impedance measurement furthermore allows a non-invasive determination of the glucose concentration, as described in detail in the cited WO 2008/061788 A1, if necessary in combination with other measurement modalities.

It is advantageous that the pressure sensor can be mechanically coupled with a measurement electrode of the EKG unit and/or of the bioelectrical impedance measurement unit, and thereby detect the pressure locally exerted on the pressure sensor by the body tissue to be examined, by way of the measurement electrode. In this manner, the pressure sensor can be integrated in particularly elegant manner. The measurement electrode and the pressure sensor use one and the same contact surface on the body for measurement signal detection. In this connection, to allow the pressure or force measurement, the measurement electrode should be movably (e.g. resiliently) mounted on the diagnostic sensor unit. The actual pressure sensor can then comprise a piezoresistive element, for example. The piezoresistive element can advantageously be disposed below the measurement electrode of the EKG unit and/or of the bioelectrical impedance unit. In this manner, the force or pressure exerted on the electrode is directly converted to an electrical measurement signal.

The measurement device according to the invention offers a great number of advantages for the determination of one or more physiological parameters. The known sensor systems in medical measurement devices (see WO 2008/061788 A1) are supplemented in practical manner. The measurement accuracy can be increased. The pressure sensor can be used as a reference, in order to uncover the errors of the measurement signals that are obtained by means of other measurement modalities. The pressure sensor furthermore has the advantage that it can be made usable with simple electronic circuits. Furthermore, pressure sensors are energy-saving and can be handled in simple manner, both in terms of production technology and in terms of application technology. Pressure sensors can be implemented in a small size and have a low error tolerance. Use of one or more pressure sensors in the measurement device according to the invention as the sole measurement modality is just as possible as use for supplementing other measurement modalities. Thus, for example, the pressure volume pulse, the pulse frequency, and further parameters connected with them can already be determined solely and exclusively from the measurement signal of the pressure sensor, which is sufficient for many practical applications. Accordingly, it is possible to do without a (more complicated) optical measurement or an EKG measurement for a great number of application purposes.

In the case of a measurement device having a diagnostic sensor unit, which device is preferably configured as described above, the evaluation unit can be set up for derivation of the at least one physiological parameter as a function of the depth in the body tissue. Methods for surface analysis and body cross-section analysis, for determination of physiological parameters, are generally known and usual. In contrast, depth analysis or depth profile analysis, in which one or more physiological parameters are determined with local resolution, particularly with depth resolution, is particularly advantageous. Particularly preferably, detection of physiological parameters with depth resolution and time dependence takes place. The data obtained in this manner make it possible to analyze physiological processes that take place in the body tissue, particularly metabolic processes, in detail, in quantitative manner, with local resolution. This in turn allows a more precise method of procedure, more comfortable for the patient or for the user of the measurement device, and a more cost-efficient and effort-efficient method of procedure in diagnosis. In the sense of the invention, a non-invasive depth analysis method for determination of physiological parameters is made available, in which method physiological parameters and corresponding biochemical processes within the human body are determined non-invasively as a function of depth. In particular, depth profile analysis allows determination of relevant physiological parameters for a determination of the composition of the body tissue, for example the tissue surrounding the capillaries, by means of use of the measurement modalities (pressure, temperature, optical measurement, bioelectrical impedance, EKG) described above, individually or in combination, in each instance. Knowledge of the composition of the body tissue in turn can be used as a parameter in the calculation for the derivation of physiological parameters from the measurement signals. A special variant of depth profile analysis is depth cross-section profile analysis. In this analysis, the cross-section of the body tissue is analyzed in the direction of depth, i.e. perpendicular to the body surface, starting from a starting point, whereby this starting point does not necessarily have to lie directly on the body surface. For example, a depth cross-section profile analysis can take place by means of bioelectrical impedance measurement, whereby the distance or the relative position of the electrode used, with regard to the body tissue being examined, the intensity of the current applied for the impedance measurement and/or the measurement frequency are varied. Thus, a measurement device according to the invention, which comprises the measurement modalities listed above, individually or in combination, is particularly well suited for depth cross-section profile analysis. Individual ones or more of the following physiological parameters can be detected with depth analysis, depth profile analysis or depth cross-section analysis, according to the invention: perfusion, blood pressure, pulse amplitude, pulse pressure, pulse width, blood amount, body surface temperature, average body temperature, arterial blood temperature, body core temperature, amount of heat transport from the body interior to the capillary ends, temperature in the region of the arterial-venous capillaries or the tissue surrounding them, pulse wave velocity, amount of heat produced by the metabolism, arterial oxygen saturation, oxygen saturation in the tissue, oxygen consumption, body water mass, proportion of fat-free mass of the body tissue, body fat proportion, glucose concentration, etc.

In this connection, and this is a significant advantage of the invention as compared with the state of the art, the hardware required for the measurement device can be structured to be very compact. The individual sensors require a construction space that can be smaller than 2 cm×2 cm×2 cm, for example. Some sensors can actually be implemented with a construction volume of less than 1 cm×1 cm×1 cm or even smaller. Even a combination of multiple measurement modalities in the measurement device according to the invention can be implemented as a compact, portable (“[in English:] handheld”) device for depth analysis or for depth profile analysis. The edge length of the device can amount to less than 10 to 20 cm, for example. Even smaller dimensions are possible. The hardware costs and thereby the diagnosis costs when using the measurement device according to the invention are clearly lower than when using conventional diagnosis methods with local resolution (e.g. computer tomography).

The underlying task mentioned above is also accomplished by a method for non-invasive determination of at least one physiological parameter, in which the pressure exerted locally on a pressure sensor by a body tissue to be examined is detected, whereby the at least one physiological parameter is derived from the detected pressure.

Exemplary embodiments of the invention will be explained in greater detail below, using the drawings. These show:

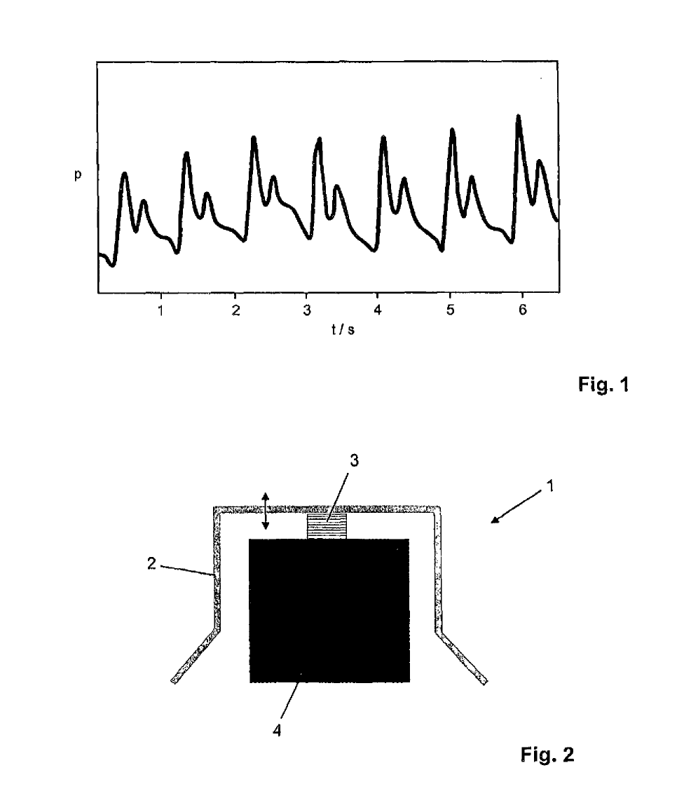

FIG. 1 measurement signal of the pressure sensor of the measurement device according to the invention, as a function of time;

FIG. 2 exemplary embodiment of a sensor unit of the measurement device according to the invention, with measurement electrode and pressure sensor;

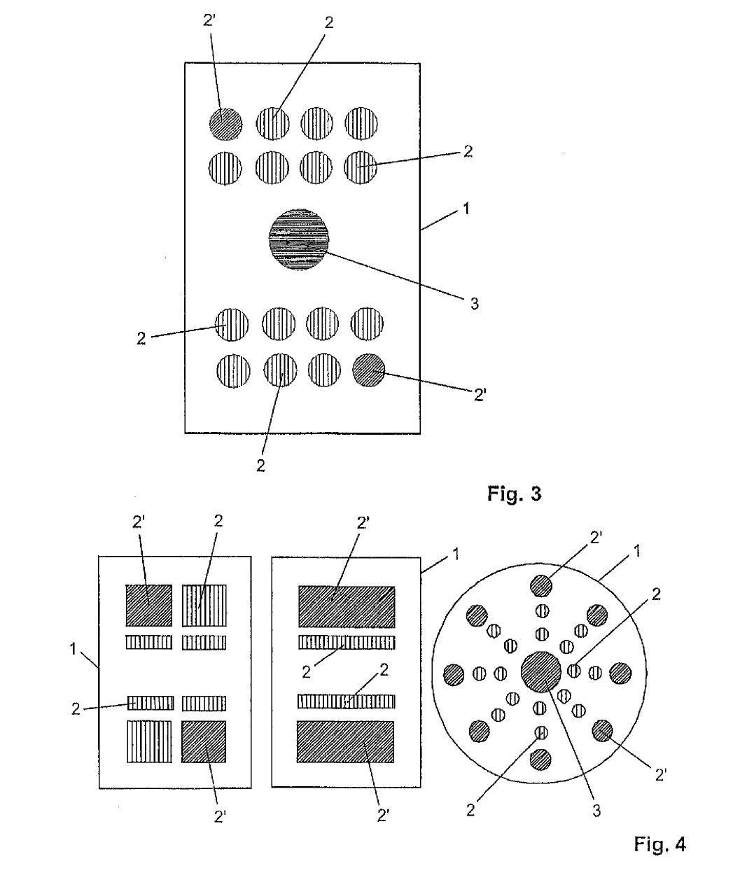

FIG. 3 another exemplary embodiment of a sensor unit of the measurement device according to the invention, with matrix-shaped placement of measurement electrodes;

FIG. 4 further exemplary embodiments with different configurations of the measurement electrodes.

FIG. 1 illustrates the derivation of the pressure volume pulse from the measurement signal of a pressure or force sensor, which is an integral part, according to the invention, of a diagnostic sensor unit. The pressure sensor lies against the body surface of a patient in the region to be examined, so that the pressure sensor detects the pressure exerted locally by the body tissue on the pressure sensor, specifically, as can be seen in FIG. 1, as a function of time t. The measurement signal is the pressure p. The time progression of the measurement signal p corresponds to the pressure volume pulse. Using the diagram, it is directly evident that physiological parameters such as pulse amplitude, pulse width, blood amount, pulse duration, perfusion, blood pressure, pulse pressure, as well as heart rate, for example, can be derived from the measurement signal. Furthermore, the pulse wave velocity can be determined. From this, in turn, other related physiological parameters can be derived. It is known, for example, that the pulse amplitude correlates with body temperature. Thus, the pressure sensor of the measurement device according to the invention can be used to determine physiological parameters that are connected with the temperature, such as body surface temperature, average body temperature, arterial blood temperature, body core temperature, the amount of heat generated by the metabolism, the amount of heat that flows from the body interior to the capillary ends, and the temperature in the region of the arterial-venous capillaries or the tissue surrounding them.

In the exemplary embodiment shown in FIG. 2, the sensor unit, shown in cross-section, is indicated as a whole with the reference number 1. The sensor unit comprises a bioelectrical impedance measurement unit having a measurement electrode 2. The measurement electrode 2 is configured as an electrically conductive plate, the surface of which, running horizontally, shown at the top in FIG. 2, is brought into contact with the body surface of the user, in order to detect electrical measurement signals (potentials) there. The measurement electrode 2 is mounted movably, namely resiliently, as indicated schematically in FIG. 2. The double arrow in FIG. 2 illustrates the mobility of the measurement electrode 2. A pressure sensor 3, for example in the form of a piezoresistive element, is disposed underneath the measurement electrode 2, which sensor detects the pressure exerted on the pressure sensor 3 by the body tissue to be examined, by way of the measurement electrode 2. In this connection, the pressure sensor 3 supports itself, at the back, on a fixed part 4.

The measurement electrode 2 and the pressure sensor 3 are connected with an evaluation unit of the measurement device according to the invention, not shown in any detail in the figures. The measurement signals of the bioelectrical impedance measurement unit and of the pressure sensor 3 are transmitted to the evaluation unit by way of this connection. The evaluation unit derives at least one physiological parameter from the measurement signals. For this purpose, the evaluation unit comprises a suitably programmed microcontroller with interfaces for digitalization of the measurement signals and with interfaces for output of the results, for example.

FIG. 3 schematically shows a top view of a surface of the sensor unit 1 that lies against the body surface of the user of the measurement device during a measurement. In the exemplary embodiment shown in FIG. 3, the pressure sensor 3 is disposed centrally. Multiple measurement electrodes 2 are disposed in the form of a matrix around the pressure sensor 3. The electrodes 2′ are feed electrodes of the bioelectrical impedance measurement unit. A measurement current is applied to the body tissue by way of the feed electrodes 2′. The potentials that occur at the body surface as a result are detected by way of the measurement electrodes 2. The matrix-shaped arrangement of the measurement and feed electrodes 2 and 2′ allows a depth profile analysis of physiological parameters, according to the invention. Depending on the relative position of the feeding and measuring electrodes 2 and 2′, the path of the electric current through the body tissue is different. A depth-resolved derivation of physiological parameters can take place by a comparison of the detected potentials, as explained in greater detail above. In the exemplary embodiment shown, a total of 16 electrodes 2 and 2′ is provided. A significantly greater number of measurement electrodes can be practical if a higher resolution is desired in the depth profile analysis, for example.

FIG. 4 shows further exemplary embodiments. In the two left configurations of FIG. 4, at least one pressure sensor is disposed underneath at least one of the electrodes 2, 2′, as shown in FIG. 2. In the variant shown on the right in FIG. 4, the electrodes 2 and 2′ are disposed in a radial configuration around the central pressure sensor 3. All the configurations are suitable for depth-resolved determination of physiological parameters, as explained above.

1. Measurement device for non-invasive determination of at least one physiological parameter, having a diagnostic sensor unit (1) for generating measurement signals, and having an evaluation unit for processing the measurement signals wherein
the diagnostic sensor unit (1) comprises at least one pressure sensor (3) that detects the pressure exerted locally on the pressure sensor (3) by a body tissue to be examined, wherein the evaluation unit is set up for derivation of at least one physiological parameter from the measurement signal of the pressure sensor (3).

2. Measurement device according to claim 1, wherein the evaluation unit is set up for deriving at least one of the following physiological parameters from the time progression of the measurement signal of the pressure sensor (3): pressure volume pulse, pulse amplitude, pulse width, pulse duration, perfusion, blood pressure, pulse pressure, heart rate, pulse wave velocity, body temperature, metabolically generated heat amount.

3. Measurement device according to claim 1, comprising means for pressing the pressure sensor (3) against the surface of the body tissue.

4. Measurement device according to claim 1, wherein the sensor unit (1) comprises an optical measurement unit that comprises at least one radiation source for irradiation of the body tissue and at least one radiation sensor for detection of the radiation scattered and/or transmitted by the body tissue, wherein the evaluation unit is set up for derivation of the at least one physiological parameter at least from the measurement signals of the pressure sensor (3) and of the optical measurement unit.

5. Measurement device according to claim 1, wherein the sensor unit (1) comprises a temperature sensor, wherein the evaluation unit is set up for derivation of the at least one physiological parameter at least from the measurement signals of the pressure sensor and of the temperature sensor.

6. Measurement device according to claim 1, wherein the sensor unit (1) comprises an EKG unit for detection of an EKG signal by way of two or more EKG electrodes, wherein the evaluation unit is set up for derivation of the at least one physiological parameter at least from the measurement signals of the pressure sensor and of the EKG unit.

7. Measurement device according to claim 1, wherein the sensor unit (1) comprises a bioelectrical impedance measurement unit, wherein the evaluation unit is set up for derivation of the at least one physiological parameter at least from the measurement signals of the pressure sensor (3) and of the bioelectrical impedance measurement unit.

8. Measurement device according to claim 6, wherein the pressure sensor (3) is mechanically coupled with a measurement electrode (2) of the EKG unit and/or of the bioelectrical impedance measurement unit, and thereby detects the pressure locally exerted on the pressure sensor (3) by the body tissue to be examined, by way of the measurement electrode (2).

9. Measurement device according to claim 8, wherein the measurement electrode (2) is movably mounted on the diagnostic sensor unit (1).

10. Measurement device according to claim 1, wherein the pressure sensor (3) comprises at least one piezoresistive element.

11. Measurement device according to claim 1, wherein the evaluation unit is set up for derivation of the glucose concentration from the measurement signals.

12. Measurement device according to claim 1, wherein the evaluation unit is set up for derivation of the at least one physiological parameter as a function of the depth in the body tissue.

13. Method for non-invasive determination of at least one physiological parameter, wherein
the pressure exerted locally on a pressure sensor (3) by a body tissue to be examined is detected, wherein the at least one physiological parameter is derived from the detected pressure.

14. Method according to claim 13, wherein at least one of the following physiological parameters is derived from the time progression of the measurement signal of the pressure sensor (3): pressure volume pulse, pulse amplitude, pulse width, pulse duration, perfusion, blood pressure, pulse pressure, heart rate, pulse wave velocity, body temperature, metabolically generated heat amount.

15. Method for non-invasive determination of at least one physiological parameter, particularly according to claim 13, wherein the derivation of the at least one physiological parameter takes place as a function of the depth in the body tissue.

16. Method according to claim 15, wherein measurement signals are generated by means of a diagnostic sensor unit, from which signals the at least one physiological parameter is derived as a function of the depth in the body tissue, wherein the sensor unit comprises:
at least one pressure sensor (3) that detects the pressure exerted locally on the pressure sensor (3) by a body tissue to be examined, and/or
an optical measurement unit that comprises at least one radiation source for irradiation of the body tissue and at least one radiation sensor for detection of the radiation scattered and/or transmitted by the body tissue, and/or
a temperature sensor, and/or
an EKG unit for detection of an EKG signal by way of two or more EKG electrodes, and/or
a bioelectrical impedance measurement unit.
